(19) United States
(12) Patent Application Publication
(10) Pub. No.: US 2025/0297946 A1
(43) Pub. Date: Sep. 25, 2025

(54) Spectroscopic Apparatus, Spectroscopic Apparatus Calibration Method, And Spectroscopic Method

(71) Applicant: SEIKO EPSON CORPORATION, Tokyo (JP)

(72) Inventors: Nobuhito HAYASHI, Chino (JP); Kohei YAMADA, Shiojiri (JP)

(21) Appl. No.: 19/086,271

(22) Filed: Mar. 21, 2025

(30) Foreign Application Priority Data
Mar. 22, 2024 (JP) 2024-046163

(51) Int. Cl. G01N 21/31; G01B 11/02; G01N 33/00

(52) U.S. Cl. CPC G01N 21/31; G01B 11/02; G01N 33/0027; G01N 2201/06113; G01N 2201/0636

(57) ABSTRACT

A spectroscopic apparatus including an analysis optical system, a length measuring optical system, and a calculation apparatus and performing spectroscopic analysis of a sample, the analysis optical system including a moving mirror having a first reflection surface and a second reflection surface, the moving mirror configured to be translated, a gas cell configured to encapsulate a gas and add a light absorption signal to the analysis light, and a first light receiver configured to receive the analysis light containing a sample derived signal, a first modulation signal, and the light absorption signal, the length measuring optical system including a length measuring optical system configured to acquire a displacement signal corresponding to the position of the moving mirror from laser light reflected off the second reflection surface, the calculation apparatus including a moving mirror position calculator, a light intensity calculator, a Fourier transformer, and a moving mirror position correction section.

The present application is based on, and claims priority from JP Application Serial Number 2024-046163, filed Mar. 22, 2024, the disclosure of which is hereby incorporated by reference herein in its entirety.

### BACKGROUND

### 1. Technical Field

The present disclosure relates to a spectroscopic apparatus, a spectroscopic apparatus calibration method, and a spectroscopic method.

### 2. Related Art

WO 2019/009404 discloses an optical module used for a spectroscopic analysis in which information on the spectrum of light emitted or absorbed by a sample is acquired and components and other factors in the sample is analyzed based on the spectral information. The optical module includes a mirror unit, a beam splitter unit, a light incident section, a first photodetector, a second light source, and a second photodetector. The mirror unit includes a movable mirror that moves in a predetermined direction and a fixed mirror fixed at a certain position. In the thus configured optical module, the beam splitter unit, the movable mirror, and the fixed mirror constitute an interference optical system that measurement target light and laser light enter.

The measurement target light incident from a first light source via a measurement target travels via the light incident section and is split by the beam splitter unit. One of the two portions into which the measurement target light is split is reflected off the movable mirror and returns to the beam splitter unit. The remaining other of the two portions into which the measurement target light is split is reflected off the fixed mirror and returns to the beam splitter unit. The one portion and the other portion of the measurement target light having returned to the beam splitter unit are detected as interference light by the first photodetector.

The laser light output from the second light source is split by the beam splitter unit. One of the two portions into which the laser light is split is reflected off the movable mirror and returns to the beam splitter unit. The remaining other of the two portions into which the laser light is split is reflected off the fixed mirror and returns to the beam splitter unit. The one portion and the other portion of the laser light having returned to the beam splitter unit are detected as interference light by the second photodetector.

In the thus configured optical module, the position of the movable mirror is measured based on the result of the detection of the laser interference light. Spectroscopic analysis of the measurement target can then be performed based on the result of the measurement of the position of the movable mirror and the result of the detection of the measurement target interference light. Specifically, a waveform called an interferogram is produced by determining the intensity of the measurement target light at each position of the movable mirror. A spectral pattern for the measurement target can be determined by performing Fourier transform on the interferogram. The optical module described in WO 2019/009404 is therefore used in a Fourier transform infrared spectroscopic analyzer (FTIR).

WO 2019/009404 is an example of the related art.

In a Fourier transform spectroscopic analyzer, measurement precision of the position of the movable mirror (moving mirror) directly links to the accuracy of the spectral pattern on the wavenumber axis (wavelength axis). There have therefore been studies on measuring the position of the movable mirror with high precision based on a length measurement technology using laser light. As part of the technology, use of a movable mirror having two light reflecting surfaces that are front and rear surfaces has been studied. To accurately measure the amount of change in the optical path length of the measurement target light by using the laser light, it is necessary to sufficiently increase the parallelism between the light reflecting surface that reflects the measurement target light and the light reflecting surface that reflects the laser light.

It is, however, not easy to increase the parallelism between the two reflection surfaces, and a length measurement error occurs when the parallelism is poor. The length measurement error causes a decrease in the accuracy of the spectral pattern acquired from the measurement target on the wavenumber axis (wavelength axis).

In view of the fact described above, it is a challenge to provide a spectroscopic apparatus capable of compensating for a decrease in the length measurement precision and generating a high-precision spectral pattern even when the two light reflecting surfaces of the moving mirror have poor parallelism.

### SUMMARY

A spectroscopic apparatus according to an example to which the present disclosure is applied is
a spectroscopic apparatus including an analysis optical system, a length measuring optical system, and a calculation apparatus and performing spectroscopic analysis of a sample,
the analysis optical system including
a moving mirror having a first reflection surface configured to reflect analysis light output from a first light source and add a first modulation signal to the analysis light, and a second reflection surface located at a side opposite the first reflection surface, the moving mirror configured to be translated,
a gas cell configured to encapsulate a gas that absorbs light having a predetermined wavelength, and add a light absorption signal to the analysis light when the analysis light enters the gas cell, and
a first light receiver configured to receive the analysis light containing sample derived signal generated by a reaction between the analysis light and the sample, the first modulation signal, and the light absorption signal, and output a first light reception signal,
the length measuring optical system including
a second light source configured to output laser light, and
a length measuring optical system configured to irradiate the second reflection surface with the laser light and acquire a displacement signal corresponding to a position of the moving mirror from the laser light reflected off the second reflection surface,
the calculation apparatus including
a moving mirror position calculator configured to generate a moving mirror position signal based on the displacement signal,
a light intensity calculator configured to generate a waveform indicating an intensity of the first light reception signal at each position of the moving mirror based on the first light reception signal and the moving mirror position signal,
a Fourier transformer configured to perform Fourier transform on the waveform to generate a spectral pattern containing a peak corresponding to the light absorption signal, and
a moving mirror position correction section configured to calculate a correction value used to correct the moving mirror position signal based on a position of the peak.

A spectroscopic apparatus calibration method according to another example to which the present disclosure is applied is a spectroscopic apparatus calibration method for calibrating a spectroscopic apparatus configured to perform spectroscopic analysis of a sample, the method including:
in the spectroscopic apparatus according to the example to which the present disclosure is applied, placing the gas cell in an optical path of the analysis light, causing the spectroscopic apparatus to acquire the displacement signal, and measuring the position of the moving mirror;
causing the analysis light to enter the gas cell while changing the position of the moving mirror, causing the first light receiver to receive the analysis light output from the gas cell, and outputting the first light reception signal derived from the gas cell;
generating a waveform indicating the intensity of the first light reception signal derived from the gas cell at each position of the moving mirror based on the first light reception signal derived from the gas cell and a measured value of the position of the moving mirror;
performing Fourier transform on the waveform derived from the gas cell to generate a spectral pattern containing a peak corresponding to the light absorption signal; and
calculating a correction value used to correct the measured value of the position of the moving mirror based on a difference between a wavelength at which the peak is located and a basic wavelength absorbed by the gas cell.

A spectroscopic method according to another example to which the present disclosure is applied includes:
executing the spectroscopic apparatus calibration method according to the example to which the present disclosure is applied; and
in the spectroscopic apparatus, placing the sample in the optical path of the analysis light, then acquiring a spectral pattern containing information derived from the sample, and correcting the spectral pattern containing the information derived from the sample based on the correction value.

### BRIEF DESCRIPTION OF THE DRAWINGS

FIG. 1 is a schematic configuration diagram showing a spectroscopic apparatus according to a first embodiment.

FIG. 2 is a cross-sectional view showing an example of the configuration of a moving mirror in FIG. 1.

FIG. 3 is a cross-sectional view showing another example of the configuration of the moving mirror in FIG. 1.

FIG. 4 is a diagrammatic view showing key parts of an analysis optical system, a length measuring optical system, a signal generator, and a calculation apparatus in FIG. 1.

FIG. 5 shows an example of a first light reception signal and a second light reception signal acquired by the spectroscopic apparatus shown in FIG. 1.

FIG. 6 shows an example of an interferogram.

FIG. 7 is a partially enlarged view of the second light reception signal shown in FIG. 5.

FIG. 8 is an energy level diagram showing an ultrafine structure of a cesium atom at the Cs (D1) line.

FIG. 9 is an absorption spectrum produced by absorption of the Cs (D1) line shown in FIG. 8.

FIG. 10 is an example of a spectral pattern produced by the spectroscopic apparatus shown in FIG. 4.

FIG. 11 is a flowchart for illustrating a spectroscopic method including a spectroscopic apparatus calibration method according to the first embodiment.

FIG. 12 is a schematic configuration diagram showing a spectroscopic apparatus according to a second embodiment.

FIG. 13 is a schematic configuration diagram showing a spectroscopic apparatus according to a third embodiment.

FIG. 14 is a diagrammatic view showing key parts of the analysis optical system, the length measuring optical system, the signal generator, and the calculation apparatus in FIG. 13.

FIG. 15 shows an example of the first light reception signal and a moving mirror position signal acquired by the spectroscopic apparatus shown in FIG. 13.

FIG. 16 shows graphs representing the relationship between the measurement interval at which the position of the moving mirror is measured and a maximum measured wavenumber and a minimum measured wavelength in a spectral pattern.

### DESCRIPTION OF EMBODIMENTS

A spectroscopic apparatus and a spectroscopic apparatus calibration method according to an embodiment of present disclosure will be described below in detail with reference to the accompanying drawings.

### 1. First Embodiment

A spectroscopic apparatus and a spectroscopic apparatus calibration method according to a first embodiment will first be described.

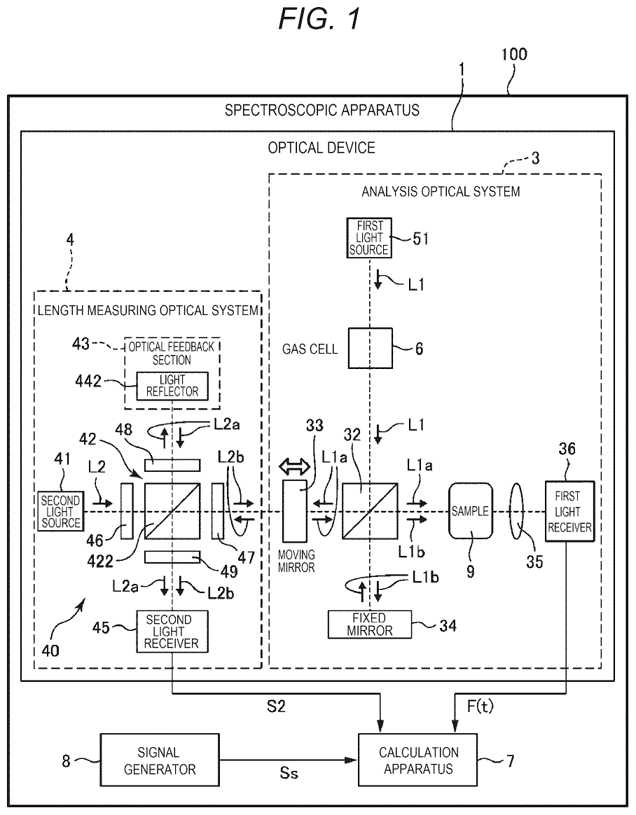

FIG. 1 is a schematic configuration diagram showing a spectroscopic apparatus 100 according to the first embodiment.

### 1.1. Spectroscopic Apparatus

In the spectroscopic apparatus 100 shown in FIG. 1, an interferogram is acquired by irradiating a sample 9, which is an object under detection, with analysis light L1 output from a first light source 51, causing the analysis light L1 emitted from the sample 9 to pass through a Michelson interference optical system, detecting a change in the intensity of the resultant interference light, and performing calculation that will be described later on the detected change. Fourier transform is performed on the acquired interferogram to produce a spectral pattern (spectral information) containing information derived from the sample 9. The spectroscopic apparatus 100 shown in FIG. 1, which selects the wavelength of the analysis light L1, is applicable, for example, to the following spectroscopic analysis of the sample 9: Fourier infrared spectroscopic analysis (FT-IR); Fourier near-infrared spectroscopic analysis (FT-NIR); Fourier visible spectroscopic analysis (FT-VIS); Fourier ultraviolet spectroscopic analysis (FT-UV); and Fourier terahertz spectroscopic analysis (FT-THz).

The spectroscopic apparatus 100 includes an optical device 1, a signal generator 8, and a calculation apparatus 7.

The optical device 1 includes an analysis optical system 3 and a length measuring optical system 4, as shown in FIG. 1.

The analysis optical system 3 irradiates the sample 9 with the analysis light L1 and splits and mixes the analysis light L1 while changing the optical path length of the analysis light L1 so that a sample derived signal derived from the sample 9 can be extracted from the analysis light L1, resulting in interference between the portions into which the analysis light L1 is split. In the length measuring optical system 4, a change in the optical path length of the analysis light L1 is measured by using length measurement light L2, which is laser light.

The signal generator 8 has the function of outputting a reference signal Ss toward the calculation apparatus 7, and may, for example, be a function generator described later. The calculation apparatus 7 has the function of determining a waveform indicating the intensity of the interference light with respect to the optical path length, that is, the interferogram described above based on a signal indicating the intensity of the interference light output from the analysis optical system 3 and a signal indicating the change in the optical path length of the light output from the length measuring optical system 4. The calculation apparatus 7 further has the function of performing Fourier transform on the interferogram to acquire the spectral pattern.

### 1.2. Optical Device

The optical device 1 will next be described.

The optical device 1 includes the analysis optical system 3 and the length measuring optical system 4, as described above.

### 1.2.1. Analysis Optical System

The analysis optical system 3 includes the first light source 51, a gas cell 6, a beam splitter 32, a moving mirror 33, a fixed mirror 34, a light collecting lens 35, and a first light receiver 36, which constitute a Michelson interference optical system. Note that in the analysis optical system 3, some of the optical elements described above may be omitted, optical elements other than those described above may be provided, or the optical elements described above may be replaced with other optical elements having the same functions.

The first light source 51 is a light source that outputs, for example, white light, that is, light having a wide wavelength range, as the analysis light L1. The wavelength band of the analysis light L1, that is, the type of the first light source 51 is selected as appropriate in accordance with the purpose of the spectroscopic analysis performed on the sample 9. When infrared spectroscopic analysis is performed, examples of the first light source 51 may include a halogen lamp, an infrared lamp, a tungsten lamp, and a black-body-radiation lamp. When visible light spectroscopic analysis is performed, the first light source 51 may, for example, be a halogen lamp. When ultraviolet spectroscopic analysis is performed, examples of the first light source 51 may include a deuterium lamp and an ultraviolet light emitting diode (UV-LED).

Note that the spectroscopic apparatus 100 can perform ultraviolet spectroscopic analysis or visible light spectroscopic analysis by selecting a wavelength longer than or equal to 100 nm but shorter than 760 nm as the wavelength of the analysis light L1. Instead, the spectroscopic apparatus 100 can perform infrared spectroscopic analysis or near-infrared spectroscopic analysis by selecting a wavelength longer than or equal to 760 nm but shorter than 20 μm as the wavelength of the analysis light L1. Still instead, the spectroscopic apparatus 100 can perform terahertz-wave spectroscopic analysis by selecting a wavelength longer than or equal to 30 μm but shorter than 3 mm as the wavelength of the analysis light L1.

Note that the first light source 51 may be provided outside the spectroscopic apparatus 100. In this case, the analysis light L1 output from the first light source 51 provided outside the spectroscopic apparatus 100 only needs to be introduced into the spectroscopic apparatus 100. The present embodiment, in which the spectroscopic apparatus 100 includes the first light source 51 so that the first light source 51 and the beam splitter 32 can be aligned with each other with particularly increased precision, can minimize loss of the analysis light L1 caused by alignment failure therebetween.

The analysis light L1 is collimated by using a lens, a concave mirror, or any other optical element that is not shown, and then enters the gas cell 6. The gas cell 6 encapsulates a gas that absorbs light having a predetermined wavelength. When the analysis light L1 enters the gas cell 6, a light absorption signal is added to the analysis light L1. The light absorption signal is the gas absorption of light having a specific wavelength. The gas cell 6 will be described later in detail.

The analysis light L1 having passed through the gas cell 6 enters the beam splitter 32. The beam splitter 32 is a non-polarizing beam splitter that splits the analysis light L1 into two, analysis light L1a and analysis light L1b. Specifically, the beam splitter 32 has the function of splitting the analysis light L1 into two by reflecting part of the analysis light L1 toward the moving mirror 33 as the analysis light L1a and transmitting the other part of the analysis light L1 toward the fixed mirror 34 as the analysis light L1b.

Examples of the type of the beam splitter 32 may include a plate-shaped element and a stack-shaped element in addition to a prism-shaped element (cube-shaped element) shown in FIG. 1. Since using the plate-shaped beam splitter 32 causes wavelength dispersion between the analysis light L1a and the analysis light L1b, a wavelength dispersion compensator may be disposed between the beam splitter 32 and the fixed mirror 34 as required.

The beam splitter 32 transmits the analysis light L1a reflected off the moving mirror 33 toward the first light receiver 36, and reflects the analysis light L1b reflected off the fixed mirror 34 toward the first light receiver 36. The beam splitter 32 therefore has the function of mixing the analysis light L1a and the analysis light L1b, into which the analysis light L1 is split.

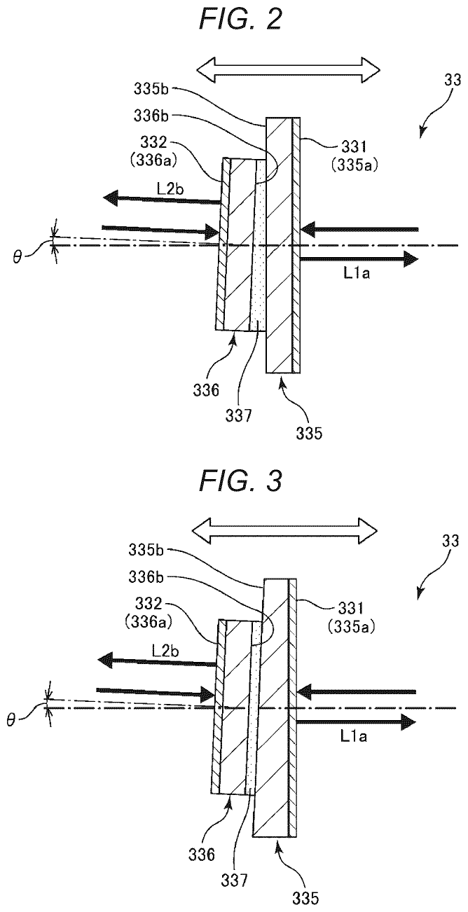

FIGS. 2 and 3 are cross-sectional views each showing an example of the configuration of the moving mirror 33 in FIG. 1.

The moving mirror 33 has a first reflection surface 331 and a second reflection surface 332, which are front and rear surfaces, and the moving mirror 33 is translated, as shown in FIGS. 2 and 3.

The moving mirror 33 moves relative to the beam splitter 32 in the direction in which the analysis light Lla is incident, and reflects the analysis light L1a at the first reflection surface 331. The phase of the analysis light L1a reflected off the moving mirror 33 changes in accordance with the position of the moving mirror 33. The moving mirror 33 thus adds a first modulation signal to the analysis light L1a. The first modulation signal is a change in the phase added to the analysis light L1a in accordance with the position of the moving mirror 33.

The position of the moving mirror 33 is measured by the length measuring optical system 4, which will be described later. The laser light for length measurement output from the length measuring optical system 4 is reflected off the second reflection surface 332. The length measuring optical system 4 measures the position of the moving mirror 33 based on the reflected laser light.

A moving mechanism that is not shown but moves the moving mirror 33 is not limited to a specific mechanism, and may, for example, be a uniaxial linear stage, a piezoelectric driving apparatus, a micro-actuator using a micro-electro-mechanical-systems (MEMS) technology.

The moving mirror 33 has the first reflection surface 331 and the second reflection surface 332, which are front and rear surfaces, as shown in FIGS. 2 and 3. Specifically, the moving mirror 33 shown in FIGS. 2 and 3 includes a first mirror member 335 and a second mirror member 336. The first reflection surface 331 is a front surface 335a of the first mirror member 335, and the second reflection surface 332 is a front surface 336a of the second mirror member 336, which is attached to a rear surface 335b of the first mirror member 335. The rear surface 335b of the first mirror member 335 and a rear surface 336b of the second mirror member 336 are bonded to each other via an adhesive layer 337.

According to the configuration described above, in which the moving mirror 33 is configured with the combination of the two mirror members, the reflectance is readily increased at both the first reflection surface 331 and the second reflection surface 332. The configuration described above increases the S/N ratio (signal-to-noise ratio) of the interference light containing the analysis light L1 reflected off the first reflection surface 331. Similarly, the S/N ratio of the interference light containing the laser light for length measurement reflected off the second reflection surface 332 is also increased.

The first reflection surface 331 and the second reflection surface 332 of the moving mirror 33 are required to be parallel to each other. In practice, however, there is a decrease in the parallelism due to an error in the manufacture of the moving mirror 33, precision of the members involved, and the like. Such a decrease in the parallelism reduces the measurement precision of the position of the moving mirror 33.

Specifically, when the first reflection surface 331 and the second reflection surface 332 are not parallel to each other, the optical axis of the analysis light L1 incident on the first reflection surface 331 and the optical axis of the laser light for length measurement incident on the second reflection surface 332 are not parallel. The non-parallelism causes a discrepancy between the actual traveling distance of the moving mirror 33 and the measured traveling distance of the moving mirror 33 measured by the length measuring optical system 4. Such a discrepancy in the length measurement causes a decrease in the precision of the spectral pattern acquired by the spectroscopic apparatus 100.

FIG. 2 diagrammatically shows a decrease in the parallelism due to an error in the manufacture of the moving mirror 33.

The adhesive layer 337 shown in FIG. 2 has a variation in thickness. When the thickness of the adhesive layer 337 varies, the parallelism between the first reflection surface 331 and the second reflection surface 332 decreases. As a result, for example, the optical axis of the length measurement light L2 deviates by an angle θ from the optical axis of the analysis light L1a.

In FIG. 3, the first mirror member 335 and the second mirror member 336 themselves have poor dimensional precision, specifically, the front surface 335a and the rear surface 335b of the first mirror member 335, and the front surface 336a and the rear surface 336b of the second mirror member 336 each have poor parallelism. As a result, for example, the optical axis of the length measurement light L2 deviates by an angle θ from the optical axis of the analysis light L1a.

It is not easy to suppress the error in the manufacture of the moving mirror 33 shown in FIG. 2 and increase the dimensional precision of the members shown in FIG. 3 because such suppression and increase lead to an increase in the manufacturing cost of the moving mirror 33.

In view of the fact described above, in the present embodiment, the spectroscopic apparatus 100 is calibrated by using the gas cell 6, which will be described later, to compensate for a decrease in the precision of the measurement of the position of the moving mirror 33.

The fixed mirror 34, the position of which is fixed relative to the beam splitter 32, reflects the analysis light L1b. The analysis light L1b reflected off the fixed mirror 34 is mixed with the analysis light L1a in the beam splitter 32, and the mixture is received as the interference light by the first light receiver 36. In the analysis optical system 3, an optical path difference occurs between the optical path of the analysis light L1a and the optical path of the analysis light L1b in accordance with the position of the moving mirror 33. The intensity of the interference light therefore changes in accordance with the position of the moving mirror 33.

The moving mirror 33 and the fixed mirror 34 may each be a planar mirror or a retroreflective optical element such as a corner cube mirror. A metal coat made of a metal such as Al, Au, or Ag, a dielectric multilayer film, or the like may be formed at the reflection surface of each of the mirrors.

The light collecting lens 35 collects the interference light, that is, the mixture of the analysis light L1a and the analysis light L1b at the first light receiver 36.

The first light receiver 36 receives the interference light and acquires the intensity thereof. The first light receiver 36 then outputs a signal indicating a temporal change in the intensity as a first light reception signal F(t). The first light reception signal F(t) contains the sample derived signal generated by the interaction between the analysis light L1 and the sample 9, the first modulation signal described above, and the light absorption signal described above. Out of the three signals described above, the sample derived signal may, for example, be absorption of light having a specific wavelength and absorbed by the sample 9 when the analysis light L1 reacts with the sample 9.

Examples of the first light receiver 36 may include a photodiode and a phototransistor. Out of the elements described above, examples of the photodiode may include an InGaAs-based photodiode, a Si-based photodiode, and an avalanche photodiode.

Employing an element capable of acquiring a two-dimensional light intensity distribution as the first light receiver 36 allows the spectroscopic apparatus 100 to be also applied, for example, to a white-light interferometric shape measuring apparatus or an optical coherence tomography (OCT) imaging apparatus.

### 1.2.2. Length Measuring Optical System

The length measuring optical system 4 is a Michelson interference optical system and includes a second light source 41 and a length measuring optical system 40. The length measuring optical system 40 acquires a displacement signal corresponding to the position of the moving mirror 33 based on laser interference using the length measurement light L2 (laser light). The position of the moving mirror 33 can therefore be measured with precision. The length measuring optical system 40 shown in FIG. 1 includes a second light divider 42, an optical feedback section 43, and a second light receiver 45. Note that in the length measuring optical system 4, some of the optical elements described above may be omitted, optical elements other than the optical elements described above may be provided, or the optical elements described above may be replaced with other optical elements having the same functions.

The second light source 41 is preferably a light source that outputs light having a narrow spectral linewidth. Examples of the second light source 41 may include a gas laser such as a He—Ne laser and an Ar laser; a semiconductor laser element such as a distributed feedback laser diode (DFB-LD), a fiber Bragg grating laser diode (FBG-LD), a vertical cavity surface emitting laser (VCSEL), and a Fabry-Perot laser diode (FP-LD); and a crystal laser made, for example, of yttrium aluminum garnet (YAG).

It is particularly preferable that the second light source 41 is a semiconductor laser element. The sizes and weights of the optical device 1 and the spectroscopic apparatus 100 can therefore be reduced.

The second light divider 42 includes a beam splitter 422, a half-wave plate 46, a quarter-wave plate 47, a quarter-wave plate 48, and an analyzer 49.

The beam splitter 422 is a polarizing beam splitter that transmits P-polarized light and reflects S-polarized light. The half-wave plate 46 is disposed with the optical axis thereof rotated with respect to the polarization axis of the length measurement light L2. In the configuration described above, the length measurement light L2 passes through the half-wave plate 46, becomes linearly polarized light containing P-polarized light and S-polarized light, and is split by the beam splitter 422 into two, the P-polarized light and the S-polarized light.

Length measurement light L2a, which is S-polarized light, is converted into circularly polarized light by the quarter-wave plate 48 and enters the optical feedback section 43. The optical feedback section 43 reflects the length measurement light L2a to cause it to be fed back to the beam splitter 422. In this process, the length measurement light L2a is converted into P-polarized light by the quarter-wave plate 48.

Length measurement light L2b, which is P-polarized light, is converted into circularly polarized light by the quarter-wave plate 47, and is incident on the moving mirror 33. The moving mirror 33 reflects the length measurement light L2b. The phase of the length measurement light L2b therefore changes in accordance with the position of the moving mirror 33. The moving mirror 33 thus adds a displacement signal to the length measurement light L2b. The length measurement light L2b reflected off the moving mirror 33 returns to the beam splitter 422. In this process, the length measurement light L2b is converted into S-polarized light by the quarter-wave plate 47.

The beam splitter 422 transmits the length measurement light L2a fed back from the optical feedback section 43 toward the second light receiver 45, and reflects the length measurement light L2b reflected off the moving mirror 33 toward the second light receiver 45. The beam splitter 422 therefore has the function of mixing the length measurement light L2a and the length measurement light L2b, into which the length measurement light L2 is split, with each other. The mixture of the length measurement light L2a and the length measurement light L2b passes through the analyzer 49 and enters the second light receiver 45.

Note that a non-polarizing beam splitter may be used as the beam splitter 422 in place of the polarizing beam splitter. In this case, since the wave plate or the like is unnecessary, the number of parts can be reduced, so that the size of the optical device 1 can be reduced.

The optical feedback section 43 includes a light reflector 442, reflects the light reflected off the beam splitter 422 and incident on the light reflector 442, and causes the light to be fed back to the beam splitter 422. The light reflector 442 is configured, for example, with a mirror. The thus configured light reflector 442 can contribute to simplification of the configuration of the optical feedback section 43, and in turn reduction in the size of the optical device 1.

The second light receiver 45 receives the mixture of the length measurement light L2a and the length measurement light L2b as interference light, and acquires the intensity thereof. The second light receiver 45 then outputs a signal indicating a temporal change in the intensity as a second light reception signal S2. The second light reception signal S2 contains the displacement signal indicating the displacement of the moving mirror 33. The displacement signal is the change in the phase added to the length measurement light L2b in accordance with the position of the moving mirror 33. The length measuring optical system 40 thus acquires the displacement signal indicating the position of the moving mirror 33.

Examples of the second light receiver 45 may include a photodiode and a phototransistor.

The analysis optical system 3 and the length measuring optical system 4 have been described above, and it is preferable that an antireflection treatment is applied to an optical element on which light needs to be incident out of the optical elements provided in the two optical systems. The S/N ratios of the first light reception signal F(t) and the second light reception signal S2 can thus be increased.

### 1.3. Signal Generator

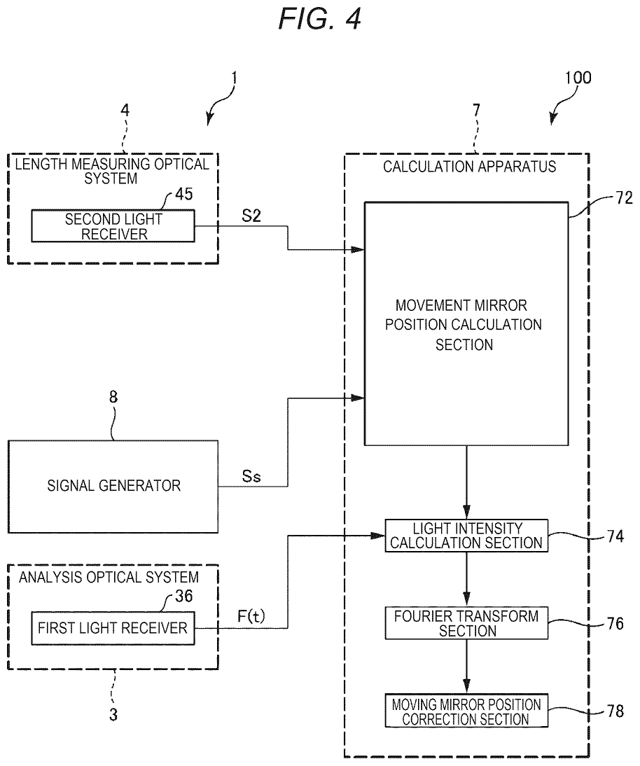

FIG. 4 is a diagrammatic view showing key parts of the analysis optical system 3, the length measuring optical system 4, the signal generator 8, and the calculation apparatus 7 in FIG. 1.

The signal generator 8 shown in FIG. 4 generates a periodic signal and outputs the periodic signal as the reference signal Ss. Examples of the signal generator 8 may include a function generator, a signal generator, and a numerically controlled signal generator. The calculation apparatus 7, which will be described later, generates a moving mirror position signal X(t) based on the reference signal Ss and the displacement signal described above.

### 1.4. Calculation Apparatus

The calculation apparatus 7 shown in FIG. 4 includes a moving mirror position calculator 72, a light intensity calculator 74, a Fourier transformer 76, and a moving mirror position correction section 78. The functions provided by the functional sections described above are realized, for example, by hardware including a processor, a memory, an external interface, an input section, and a display section. The functions are specifically realized by the processor reading and executing a program stored in the memory. Note that the elements that constitute the functional sections can communicate with each other via an external bus.

Examples of the processor may include a central processing unit (CPU) and a digital signal processor (DSP). Note that the configuration in which any of the processors described above executes software may be replaced with a configuration in which a field-programmable gate array (FPGA), an application-specific integrated circuit (ASIC), or the like realizes the functions described above.

Examples of the memory may include a hard disk drive (HDD), a solid-state drive (SSD), an electrically erasable programmable read-only memory (EEPROM), a read-only memory (ROM), and a random access memory (RAM).

Examples of the external interface may include a digital input and output port such as a universal serial bus (USB), and an Ethernet (registered trademark) port.

Examples of the input section may include various input apparatuses such as a keyboard, a mouse, a touch panel, and a touchpad. Examples of the display section may include a liquid crystal display panel and an organic electro-luminescence (EL) display panel. The input section and the display section may be provided as necessary, and may be omitted.

### 1.4.1. Moving Mirror Position Calculator

The moving mirror position calculator 72 generates, based on the reference signal Ss output from the signal generator 8, the moving mirror position signal X(t) from the second light reception signal S2 containing the displacement signal indicating the displacement of the moving mirror 33. When the moving mirror 33 moves, the intensity of the interference light in the length measuring optical system 4 changes accordingly. In this case, the second light reception signal S2 is, for example, a signal having an amplitude that periodically changes in accordance with the condition under which the interference occurs. The displacement of the moving mirror 33 can be calculated based on the change in the amplitude of the second light reception signal S2, so that the moving mirror position signal X(t) is determined.

### 1.4.2. Light Intensity Calculator

The light intensity calculator 74 generates, based on the first light receiving signal F(t) and the movement mirror position signal X(t), the waveform representing the intensity of the interference light (interferogram F(x)) with respect to the position of the moving mirror 33.

The first light reception signal F(t) contains the sample derived signal, the first modulation signal, and the light absorption signal, as described above. The light intensity calculator 74 generates the interferogram F(x) based on the position of the moving mirror 33, which is determined from the moving mirror position signal X(t), and the intensity of the first light reception signal F(t). The interferogram F(x) is expressed by a function of an optical path difference between the light reflected off the moving mirror 33 and the light reflected off the fixed mirror 34 and the intensity of the interference light received by the first light receiver 36 (intensity of first light reception signal F(t)) in the analysis optical system 3.

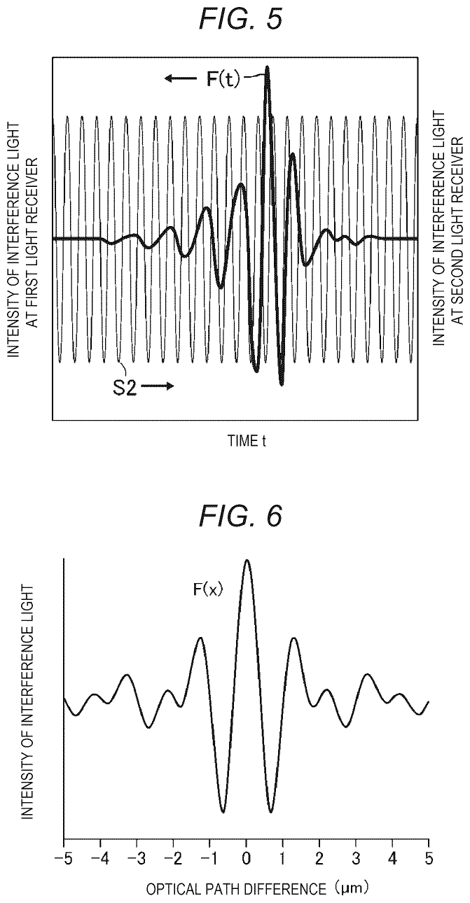

FIG. 5 shows an example of the first light reception signal F(t) and the second light reception signal S2 acquired by the spectroscopic apparatus 100 shown in FIG. 1. In FIG. 5, the horizontal axis represents time t, and the vertical axis represents the intensity of the interference light incident on the first light receiver 36 or the intensity of the interference light incident on the second light receiver 45.

FIG. 6 shows an example of the interferogram F(x). In FIG. 6, the horizontal axis represents the optical path difference in the analysis optical system 3, and the vertical axis represents the intensity of the interference light. Note that the optical path difference in the analysis optical system 3 is a difference between the optical path length between the beam splitter 32 and the moving mirror 33 and the optical path length between the beam splitter 32 and the fixed mirror 34, and the origin of the horizontal axis in FIG. 6 is the point where the optical path difference is zero.

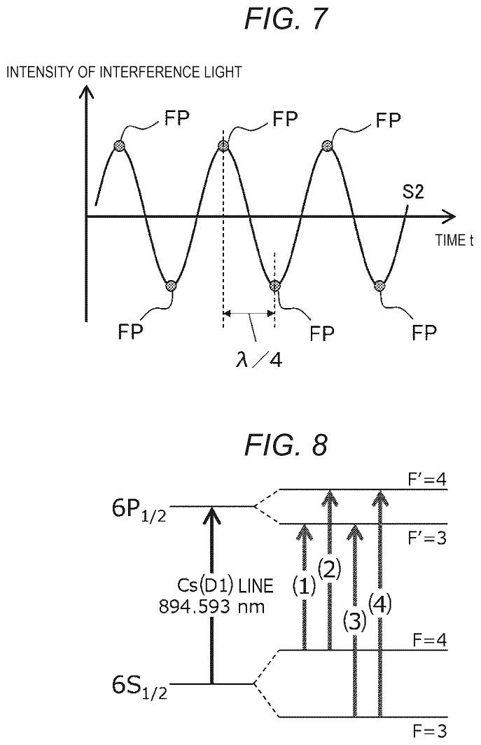

FIG. 7 is a partially enlarged view of the second light reception signal S2 shown in FIG. 5. The second light reception signal S2 shown in FIG. 7 is a signal that oscillates at a predetermined period, and a point where the amplitude is maximized is a feature point FP. The light intensity calculator 74 can associate the position of the moving mirror 33 with the intensity of the first light reception signal F(t) by extracting the intensity of the first light reception signal F(t) shown in FIG. 5 at the time points of the feature points FP. Digital data on the interferogram F(x) can thus be acquired. Note in this case that the measurement is performed based on the narrowest interval between the feature points FP, so that the narrowest interval between two points where the position of the moving mirror 33 is measured is a quarter of a wavelength A of the length measurement light L2.

### 1.4.3. Fourier Transformer

The Fourier transformer 76 performs Fourier transform on the interferogram F(x). A spectral pattern unique to the sample 9 is thus produced.

The sample derived signal generated by the analysis light L1 reacting with the sample 9 is reflected in the form of an absorption peak in the spectral pattern. The characteristics of the sample 9, for example, materials, structures, components, and the like can be analyzed based on the spectral pattern.

### 1.4.4. Moving Mirror Position Correction Section

The moving mirror position correction section 78 calculates a correction value used to correct a measured value of the position of the moving mirror by using a method that will be described later. The displacement of the moving mirror 33 contained in the moving mirror position signal X(t) can thus be brought close to a true value. As a result, the accuracy of the eventually produced spectral pattern on the wavenumber axis (wavelength axis) can be increased.

### 1.5. Gas Cell

The gas cell 6 will next be described. The gas cell 6 encapsulates a gas that absorbs light having a predetermined wavelength. Examples of the encapsulated gas may include alkali metals such as gaseous cesium and rubidium, halogens such as gaseous iodine, rare gases such as krypton, and also hydrogen cyanide and acetylene. The atoms or molecules of any of the gases described above absorb and emit the light having a predetermined wavelength. The gas cell 6 may be provided with a temperature adjusting mechanism that is not shown. The vapor pressure of the gas can thus be sufficiently increased even when the size of the gas cell 6 is further reduced. As a result, the size of the gas cell 6 can be reduced.

Table 1 below shows examples of the combination of the gas (atoms or molecules) encapsulated in the gas cell 6 and the wavelength of light with which the gas is irradiated.

TABLE 1

| Wavelength | Atoms/molecules encapsulated in gas cell |
|---|---|
| 633 nm | Iodine |
| 780 nm | ⁸⁵Rb (D2 line), ⁸⁷Rb (D2 line) |
| 795 nm | ⁸⁵Rb (D1 line), ⁸⁷Rb (D1 line) |
| 852 nm | Cs (D2 line) |
| 895 nm | Cs (D1 line) |
| 1550 nm | HCN, C₂H₂, Kr |

The wavelength of the light to be absorbed can be changed by selecting the gas encapsulated in the gas cell 6, as shown in Table 1. Note that the gas is so selected that the wavelength of the light to be absorbed overlaps with the spectrum of the light emitted from the first light source 51.

When the analysis light L1 enters the gas cell 6, the gas encapsulated in the gas cell 6 is irradiated with the analysis light L1. The atoms or molecules that constitute the gas therefore transition from the ground state to a higher-energy state (excited state) in accordance with the energy of the analysis light L1.

FIG. 8 is an energy level diagram showing an ultrafine structure of a cesium atom at the Cs (D1) line.

A cesium atom has an energy level expressed by 6S_{1/2} as the ground level, and an energy level expressed by 6P_{1/2} as an excited level, as shown in FIG. 8. The energy levels 6S_{1/2} and 6P_{1/2} each have an ultrafine structure having multiple energy levels into which the ground level is divided. Specifically, the energy level 6S_{1/2} has two ground levels expressed by F=3 and F=4. The energy level 6P_{1/2} has two excited levels expressed by F′=3 and F′=4.

A cesium atom at the ground level transitions to the excited level when absorbing, for example, the Cs (D1) line shown in FIG. 8.

For example, a cesium atom at the ground level expressed by F=4 transitions to the excited level expressed by F′=3 when absorbing the energy between the levels indicated by the arrow (1) in FIG. 8. The cesium atom further transitions to the excited level expressed by F′=4 when absorbing the energy between the levels indicated by the arrow (2) in FIG. 8.

A cesium atom at the ground level expressed by F=3 transitions to the excited level expressed by F′=3 when absorbing the energy between the levels indicated by the arrow (3) in FIG. 8. The cesium atom further transitions to the excited level expressed by F′=4 when absorbing the energy between the levels indicated by the arrow (4) in FIG. 8.

Resonance wavelengths corresponding to the transitions indicated by the arrows (1) to (4) in FIG. 8 are shown in Table 2 below.

TABLE 2

| | | Resonance wavelength |
|---|---|---|
| Label | Transition | [nm] |
| (1) | 6S_{1/2} F = 4 → 6P_{1/2} F′ = 3 | 894.6054 |
| (2) | 6S_{1/2} F = 4 → 6P_{1/2} F′ = 4 | 894.6023 |
| (3) | 6S_{1/2} F = 3 → 6P_{1/2} F′ = 3 | 894.5809 |
| (4) | 6S_{1/2} F = 3 → 6P_{1/2} F′ = 4 | 894.5779 |

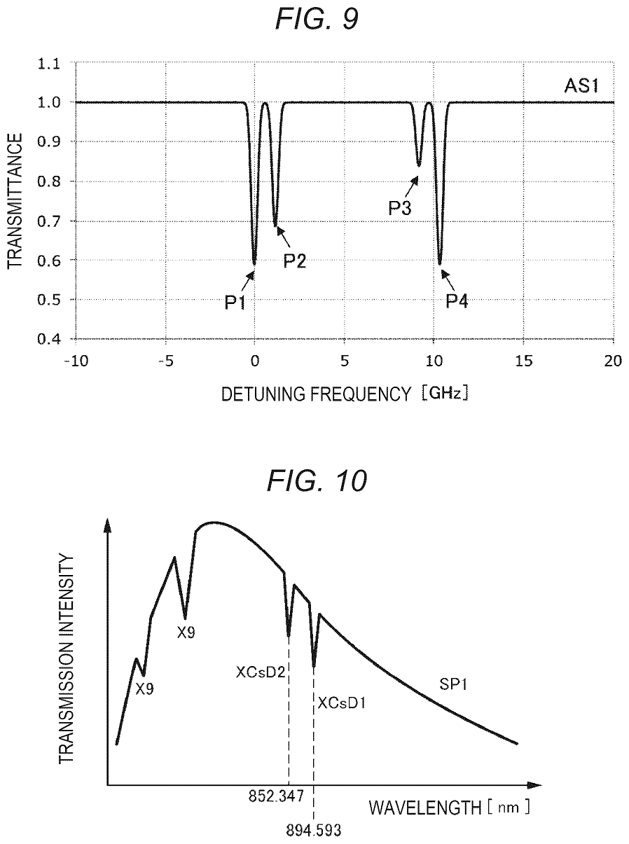

FIG. 9 is an absorption spectrum AS1 produced by absorption of the Cs (D1) line shown in FIG. 8. Four absorption peaks P1 to P4 are observed in the absorption spectrum AS1 shown in FIG. 9. The frequencies at the absorption peaks P1 to P4 correspond to four transition frequencies indicated by the arrows (1) to (4) in FIG. 8.

For example, when the analysis light L1 enters the gas cell 6, which encapsulates cesium atoms, the absorption spectrum AS1 shown in FIG. 9 is superimposed on the spectral pattern output from the Fourier transformer 76.

FIG. 10 is an example of a spectral pattern SP1 produced by the spectroscopic apparatus 100 shown in FIG. 4.

The spectral pattern SP1 shown in FIG. 10 contains absorption peaks X9 derived from the sample 9 and absorption peaks XCsD1 and XCsD2 derived from the cesium atoms. The absorption peaks X9 correspond to the sample derived signal described above. The absorption peak XCsD1 is a peak produced by the absorption of the Cs (D1) line described above. The absorption peak XCsD1 shown in FIG. 10 shows a state in which the four fine peaks shown in Table 2 do not decompose but appear as a single peak. The absorption peak XCsD2 is a peak produced by the absorption of the Cs (D2) line shown in Table 1, and shows the state again in which multiple fine peaks do not decompose but appear as a single peak. The absorption peaks XCsD1 and XCsD2 correspond to the light absorption signal described above.

The wavelengths at which the absorption peaks XCsD1 and XCsD2 contained in the spectral pattern SP1 are located each correspond to the energy between the levels described above, and are therefore extremely precise and stable. The wavelengths vary due to a change in temperature only by an order of picometers. It can therefore be said that the wavelengths at which the absorption peaks XCsD1 and XCsD2 are located have known “true values (fundamental wavelengths)”. Therefore, when there is a “wavelength discrepancy Δλ” between an actually measured value of the wavelength at which the absorption peaks XCsD1 and XCsD2 contained in the spectral pattern SP1 as a result of the analysis are each located and the true value (fundamental wavelength), the wavelength discrepancy Δλ is considered to be caused by various errors that occur in the spectroscopic apparatus 100.

Consider now a case where there is a measurement error in a measured traveling distance of the moving mirror 33 measured by the length measuring optical system 4 described above, and how the measurement error affects the spectral pattern. For example, it is assumed that a true value L [mm] of the traveling distance of the moving mirror 33 is measured, and that a measured value L(1+σ) [mm] including an error σ is acquired. In this case, the error σ causes a discrepancy between a measured value of the wavelength and a true value A thereof in the spectral pattern SP1. The discrepancy can be expressed as σλ by using the error σ. The error σ is a correction value used to produce the moving mirror position signal X(t) after the correction.

As an example, assume that the ultrafine structure contained in the absorption peak XCsD1 shown in FIG. 10 has decomposed, and consider a case where the actually measured wavelength for the ultrafine structure corresponding to the absorption peak P1 in FIG. 9 is 892.0000 nm. Since the true value of the resonance wavelength in the transition (1) is 894.6054 nm, as shown in Table 2, so that the wavelength discrepancy Δλ is Δλ=894.6054-892.0000=2.6054 [nm]. In this case, since Δλ=σλ, 2.6054=σ×894.6054 is satisfied. As a result, σ=0.002912 is satisfied. Let X′ (t) be the moving mirror position signal before the correction, and the moving mirror position signal X(t) after the correction is determined by X(t)=X′ (t)/(1+σ).

Note that the error σ may be calculated from each of the multiple absorption peaks, and then the correction may be made based on the average of the errors σ or any other suitable calculation. The method for calculating the correction value is not limited to the method described above.

The moving mirror position correction section 78 provided in the calculation apparatus 7 only needs to have the function of calculating a correction value used to correct the measured value of the position of the moving mirror 33 based on a difference between the actually measured wavelength at which the absorption peak XCsD1 is located (actually measured wavelength at which peak corresponding to light absorption signal is located) and the true value of the wavelength at which the absorption peak XCsD1 is located (fundamental wavelength absorbed by gas cell 6), and correcting the moving mirror position signal X(t) based on the correction.

In the present embodiment, the gas cell 6 is disposed between the first light source 51 and the beam splitter 32. Therefore, when the sample 9 is irradiated with the analysis light L1, the gas cell 6 is also always irradiated with the analysis light L1. Therefore, in the present embodiment, the absorption peak XCsD1 can be acquired along with the absorption peaks X9 derived from the sample 9. As a result, the correction value can be calculated simultaneously with the acquisition of the spectral pattern SP1, so that the spectroscopic apparatus 100 can be calibrated in real time, allowing particularly precise spectroscopic analysis.

Note that since the energy between the levels of the atoms or molecules encapsulated in the gas cell 6 is extremely precise and stable, the advantage described above can be provided even when the wavelength of the length measurement light L2 output from the second light source 41 has poor stability. Therefore, even when a small, inexpensive element such as a semiconductor laser element is employed as the second light source 41, it is not necessary to provide additional equipment such as a light source thermostatic system. The size, weight, power consumption, and cost of the optical device 1 can thus be reduced.

The gas cell 6 is not necessarily disposed as described above and may be disposed at any position where the analysis light L1 can enter the gas cell 6. For example, the gas cell 6 may be disposed between the beam splitter 32 and the sample 9 or between the sample 9 and the light collecting lens 35 shown in FIG. 1.

The sample 9 is also not necessarily disposed as described above. For example, the sample 9 may be disposed between the first light source 51 and the beam splitter 32 shown in FIG. 1. Furthermore, the spectroscopic apparatus 100 may be configured to acquire a reflection spectrum in place of the spectral pattern SP1 described above, which is a transmission spectrum, in accordance with the change in the position where the sample 9 is disposed.

### 1.6. Spectroscopic Method

A spectroscopic method including a spectroscopic apparatus calibration method according to the first embodiment will next be described.

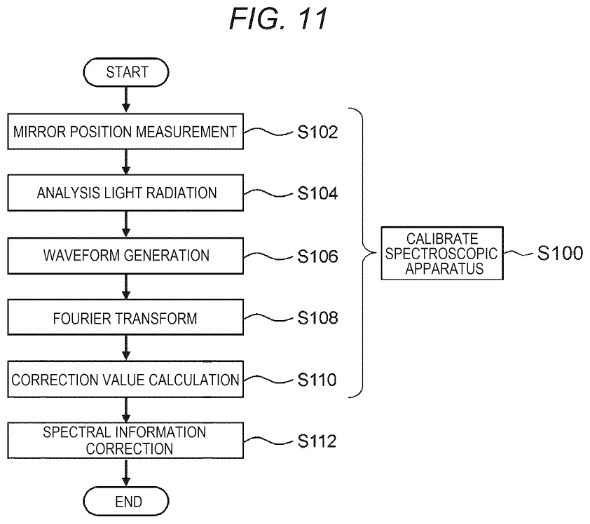

FIG. 11 is a flowchart for illustrating the spectroscopic method including the spectroscopic apparatus calibration method according to the first embodiment.

The spectroscopic apparatus calibration method shown in FIG. 11 includes a mirror position measurement step S102, an analysis light radiation step S104, a waveform generation step S106, a Fourier transform step S108, and a correction value calculation step S110. The spectroscopic method shown in FIG. 11 further includes the step of executing the calibration method (calibration step S100) and a spectral information correction step S112.

In the mirror position measurement step S102, the length measurement light L2 (laser light) is caused to enter the length measuring optical system 40 of the optical device 1, and measurement of the position of the moving mirror 33 is initiated. Acquisition of the displacement signal corresponding to the position of the moving mirror 33 is thus initiated.

In the analysis light radiation step S104, the gas cell 6 and the sample 9 are placed in the optical path of the analysis light L1, and the analysis light is caused to enter the gas cell 6 and the sample 9 with the position of the moving mirror 33 changed. The first light receiver 36 then receives the analysis light L1 output from the gas cell 6 and the sample 9, and outputs the first light reception signal F(t). Note that causing the analysis light L1 to enter the gas cell 6 and causing the analysis light L1 to be incident on the sample 9 may be performed simultaneously or at different time points. The spectroscopic apparatus 100 shown in FIG. 1 can perform the two operations simultaneously.

In the waveform generation step S106, the moving mirror position signal X(t) is generated based on the displacement signal corresponding to the position of the moving mirror 33. Thereafter, based on the first light reception signal F(t) and the moving mirror position signal X(t) (position of moving mirror 33), the interferogram F(x) (waveform indicating intensity of first light reception signal F(t) at each position of moving mirror 33) derived from both the gas cell 6 and the sample 9 is generated.

In the Fourier transform step S108, Fourier transform is performed on the interferogram F(x) to generate a spectral pattern containing the absorption peaks X9 (peaks corresponding to sample derived signal) and the absorption peak XCsD1 (peak corresponding to light absorption signal).

In the correction value calculation step S110, a correction value used to correct the measured value of the position of the moving mirror 33 is calculated based on a difference between the wavelength at the absorption peak XCsD1 and the fundamental wavelength absorbed by the gas cell 6. That is, the correction value is calculated based on the position of the absorption peak XCsD1.

In the spectral information correction step S112, the spectral pattern containing the absorption peaks X9 is corrected based on the correction value.

The above-mentioned spectroscopic apparatus calibration method can compensate for a decrease in the measurement precision (length measurement precision) of the position of the moving mirror 33 even when the first reflection surface 331 and the second reflection surface 332 of the moving mirror 33 has poor parallelism. The spectroscopic method described above, which calibrates the spectroscopic apparatus 100, can therefore generate a highly precise spectral pattern.

### 2. Second Embodiment

A spectroscopic apparatus according to a second embodiment will next be described.

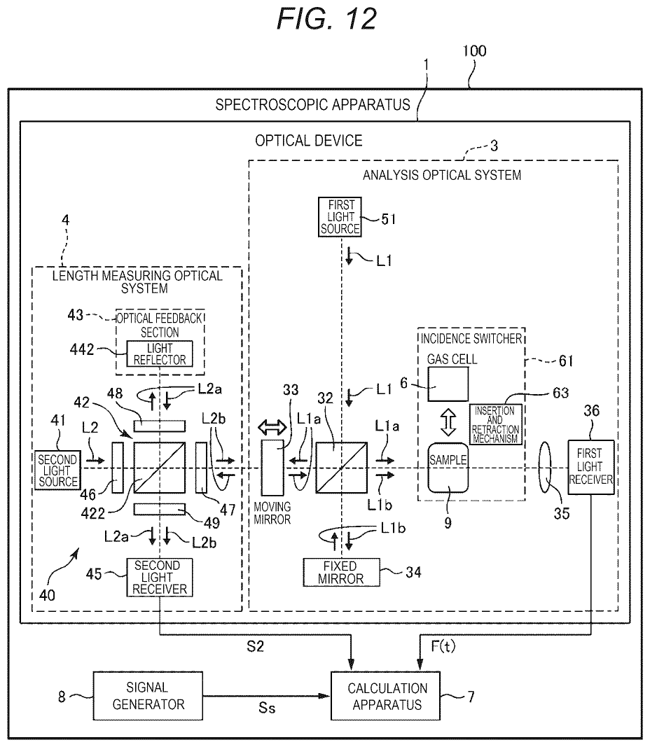

FIG. 12 is a schematic configuration diagram showing a spectroscopic apparatus 100 according to the second embodiment.

The second embodiment will be described below. In the following description, differences from the first embodiment will be primarily described, and items that are the same as those in the first embodiment will not be described. Note that the same configurations as those in the first embodiment have the same reference characters in FIG. 12.

The spectroscopic apparatus 100 shown in FIG. 12 is the same as the spectroscopic apparatus 100 shown in FIG. 1 except that the analysis optical system 3 is configured differently.

The analysis optical system 3 shown in FIG. 12 includes a sample switcher 61 provided between the beam splitter 32 and the light collecting lens 35. The sample switcher 61 includes an insertion and retraction mechanism 63, which inserts and retract the sample 9 or the gas cell 6 into and from the optical path along which the analysis light L1a and the analysis light L1b travel. The sample switcher 61 can exclusively switch a state in which the sample 9 is placed in the optical path and a state in which the gas cell 6 is placed from one to the other. As a result, the sample switcher 61 can realize the function of freely switching a spectroscopic analysis mode in which the sample 9 is spectroscopically analyzed and a calibration mode in which the spectroscopic apparatus 100 is calibrated by using the gas cell 6. The spectroscopic apparatus 100 can thus be automatically calibrated by executing the calibration mode at a necessary timing. In the spectroscopic analysis mode, since the gas cell 6 is not present in the optical path, optical loss due to the gas cell 6 can be avoided.

The second embodiment described above can also provide the same advantages provided by the first embodiment.

Note that the sample switcher 61 may be disposed between the first light source 51 and the beam splitter 32.

The sample switcher 61 shown in FIG. 12 may be omitted, and both the gas cell 6 and the sample 9 may be disposed between the beam splitter 32 and the first light receiver 36. In this case, when the sample 9 is irradiated with the analysis light L1, the gas cell 6 is also always irradiated with the analysis light L1. Therefore, in the present embodiment, the absorption peak XCsD1 can be acquired along with the absorption peaks X9 derived from the sample 9. As a result, the correction value can be calculated simultaneously with the acquisition of the spectral pattern SP1, so that the spectroscopic apparatus 100 can be calibrated in real time, allowing particularly precise spectroscopic analysis.

### 3. Third Embodiment

A spectroscopic apparatus according to a third embodiment will next be described.

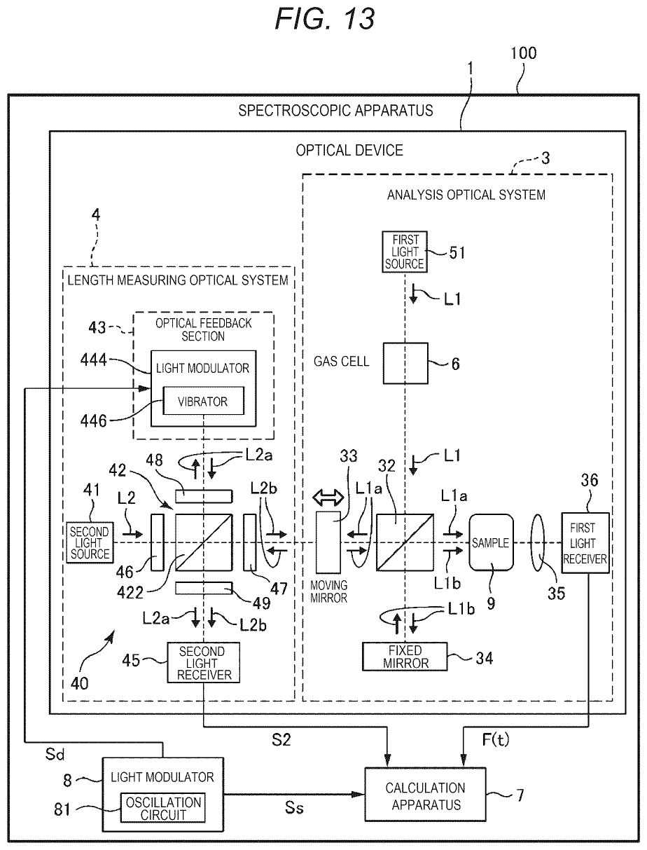

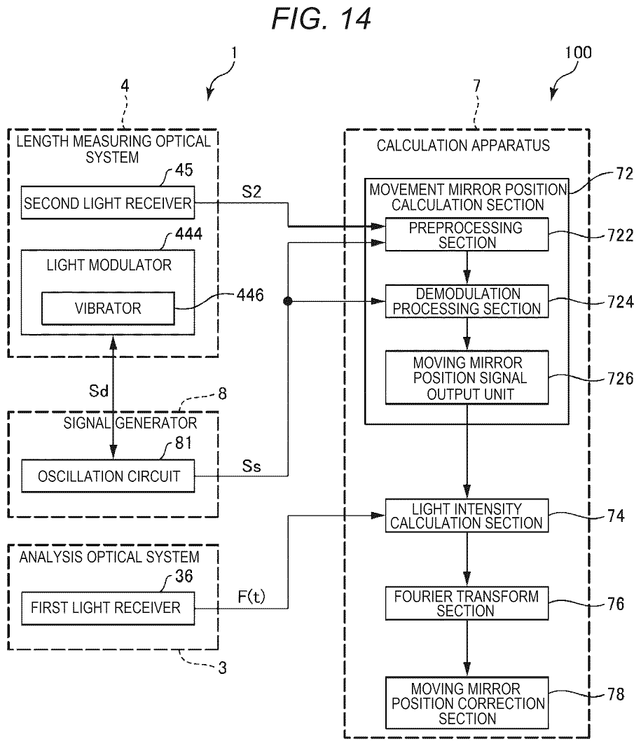

FIG. 13 is a schematic configuration diagram showing a spectroscopic apparatus 100 according to the third embodiment. FIG. 14 is a diagrammatic view showing key parts of the analysis optical system 3, the length measuring optical system 4, the signal generator 8, and the calculation apparatus 7 in FIG. 13.

The third embodiment will be described below. In the following description, differences from the first embodiment will be primarily described, and items that are the same as those in the first embodiment will not be described. Note that the same configurations as those in the first embodiment have the same reference characters in FIG. 13.

The optical feedback section 43 of the length measuring optical system 4 shown in FIG. 13 is the same as the length measuring optical system 4 shown in FIG. 1 except that the optical feedback section 43 includes a light modulator 444 in place of the light reflector 442 and the signal generator 8 is configured differently.

The light modulator 444 shown in FIG. 13 includes a vibrator 446, and shifts the frequency of the length measurement light L2a. The thus configured light modulator 444 may, for example, be the light modulator disclosed in JP-A-2022-038156. In JP-A-2022-038156, a quartz crystal AT vibrator is used as the vibrator. The vibrator 446 may instead, for example, be an SC-cut quartz crystal vibrator, a tuning-fork-type quartz crystal vibrator, a quartz crystal surface acoustic wave element.

The signal generator 8 shown in FIG. 13 has the function of generating a drive signal Sd. The signal generator 8 shown in FIG. 13 includes an oscillation circuit 81. The oscillation circuit 81 operates with the vibrator 446 serving as a signal source and generates a highly precise periodic signal. The signal generator 8 shown in FIGS. 13 and 14 causes the vibrator 446 to oscillate with the drive signal Sd, and outputs the periodic signal generated by the oscillation circuit 81 as the reference signal Ss. A second modulation signal added via the light modulator 444 driven by the drive signal Sd and the reference signal Ss are therefore under the same influence. Therefore, when the second light reception signal S2 and the reference signal Ss are subjected to the calculation in the calculation apparatus 7, the influence of disturbance contained in the two signals can be canceled out or reduced in the process of the calculation. As a result, the calculation apparatus 7 can precisely determine the position of the moving mirror 33 even when subjected to the disturbance. Furthermore, the size, weight, and power consumption of the spectroscopic apparatus 100 can be reduced.

The oscillation circuit 81 can, for example, be the oscillation circuit disclosed in JP-A-2022-038156.

The moving mirror position calculator 72 shown in FIG. 14 identifies the position of the moving mirror 33 by using optical heterodyne interferometry, and generates the moving mirror position signal X(t) based on the result of the identified position. Since the length measuring optical system 4 includes the light modulator 444, the second modulation signal can be added to the length measurement light L2a. Therefore, when the length measurement light L2a and the length measurement light L2b are caused to interfere with each other, phase information corresponding to the position of the moving mirror 33 can be acquired with higher precision from the produced interference light. The calculation apparatus 7 then determines the position of the moving mirror 33 with high precision from the phase information.

The moving mirror position calculator 72 shown in FIG. 14 includes a preprocessing section 722, a demodulation processing section 724, and a moving mirror position signal output section 726. The preprocessing section 722 and the demodulation processing section 724 may, for example, be the preprocessing section and the demodulation section disclosed in JP-A-2022-038156.

The preprocessing section 722 performs preprocessing on the second light reception signal S2 based on the reference signal Ss. The demodulation processing section 724 receives the preprocessed signal output from the preprocessing section 722 and demodulates therefrom the displacement signal according to the position of the moving mirror 33 based on the reference signal Ss.

The moving mirror position signal output section 726 generates and outputs the moving mirror position signal X(t) based on the displacement signal demodulated by the demodulation processing section 724 and indicating the displacement of the moving mirror 33. The moving mirror position signal X(t) determined by the method described above has captured the displacement of the moving mirror 33 at intervals sufficiently narrower than the wavelength of the length measurement light L2. For example, when the wavelength of the length measurement light L2 is several hundreds of nanometers, an achievable positional resolution of the position of the moving mirror 33 indicated by the displacement signal is smaller than 10 nm. In contrast, the positional resolution is limited to a quarter of the wavelength of the length measurement light L2 in the first embodiment. The light intensity calculator 74 can therefore generate digital data on the interferogram F(x) at narrower intervals than those in the first embodiment.

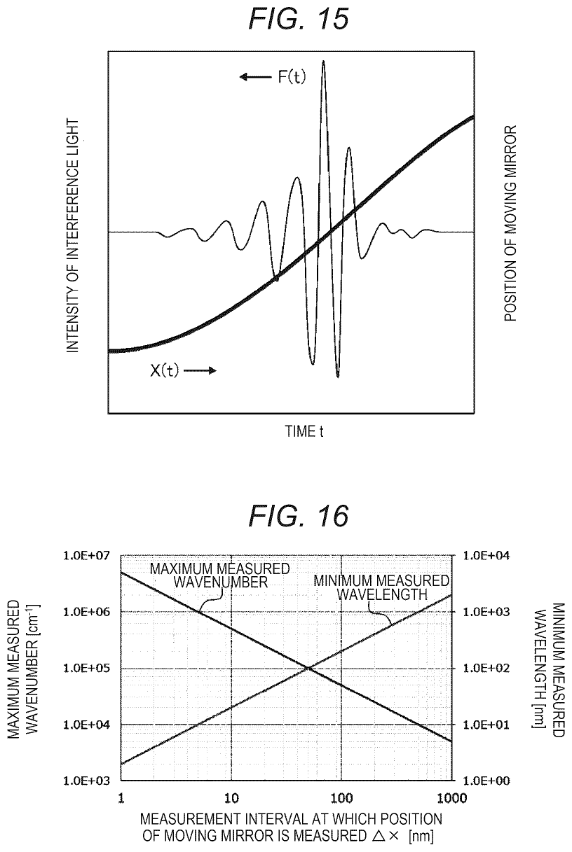

FIG. 15 shows an example of the first light reception signal F(t) and the moving mirror position signal X(t) acquired by the spectroscopic apparatus 100 shown in FIG. 13. In FIG. 15, the horizontal axis represents the time t, and the vertical axis represents the intensity of the interference light incident on the first light receiver 36 or the position of the moving mirror 33.

The moving mirror position signal X (t) shown in FIG. 15 is drawn in the form of a smooth curve as a result of continuous detection of a change in the position of the moving mirror 33, and shows that high positional resolution is achieved. Generating the interferogram F(x) based on the moving mirror position signal X(t) thus allows generation of an interferogram F(x) having a larger number of data points. The large number of data points means a narrow interval at which the interferogram F(x) has been sampled and hence high precision. Using the thus generated interferogram F(x) therefore eventually allows acquisition of a high-resolution spectral pattern.

In addition, since the sampling interval can be narrowed, a spectral pattern having a wider wavenumber range (wider wavelength range), that is, a wider-band spectral pattern can be acquired.

FIG. 16 shows graphs representing the relationship between a measurement interval Δx at which the position of the moving mirror 33 is measured and a maximum measured wavenumber and a minimum measured wavelength in a spectral pattern. The smaller the measurement interval Δx, the greater the maximum measured wavenumber and the shorter the minimum measured wavelength, as shown in FIG. 16. Narrowing the measurement interval Δx therefore allows acquisition of a spectral pattern having a wider wavenumber range (wavelength range).

Note that the vibrator 446 may be a silicon vibrator, a ceramic vibrator, a piezoelectric element, or the like in place of the quartz crystal vibrator described above. Out of the vibrators described above, the vibrator 446 is preferably a quartz crystal vibrator, a silicon vibrator, or a ceramic vibrator. The vibrators described above are those utilizing a mechanical resonance phenomenon, unlike other vibrators such as a piezoelectric element, and each therefore have a large Q-value, which readily stabilizes the natural frequency.

The silicon vibrator is a vibrator including a single crystal silicon element manufactured from a single crystal silicon substrate by using a MEMS technology, and a piezoelectric film. The term MEMS is an abbreviation for micro-electro-mechanical systems. The single crystal silicon element may, for example, have the shape of a cantilever, such as a two-leg tuning fork and a three-leg tuning fork, or the shape of a beam clamped at opposite ends. The oscillation frequency of the silicon vibrator ranges, for example, from about one kilohertz to several hundreds of megahertz.

The ceramic vibrator is a vibrator including a piezoelectric ceramic element manufactured by sintering a piezoelectric ceramic material, and electrodes. Examples of the piezoelectric ceramic material may include lead zirconate titanate (PZT) and barium titanate (BTO). The oscillation frequency of the ceramic vibrator ranges, for example, from about several hundreds of kilohertz to several tens of megahertz.

The light modulator 444 may be replaced with an acousto-optics modulator (AOM), an electro-optic modulator (EOM), or the like. The light modulator 444 including the vibrator 446 can, however, have a greatly smaller volume and weight than an AOM and an EOM. The light modulator 444 can therefore contribute to reduction in the size, weight, and power consumption of the spectroscopic apparatus 100.

### 7. Advantages Provided by Embodiments Described Above

The spectroscopic apparatus 100 according to each of the embodiments described above includes the analysis optical system 3, the length measuring optical system 4, and the calculation apparatus 7, and performs spectroscopic analysis of the sample 9.

The analysis optical system 3 includes the moving mirror 33, the gas cell 6, and the first light receiver 36. The moving mirror 33 has the first reflection surface 331, which reflects the analysis light L1 output from the first light source 51 and adds the first modulation signal to the analysis light L1, and the second reflection surface 332, which is located at the side opposite the first reflection surface 331, and the moving mirror 33 is translated. The gas cell 6, which encapsulates a gas that absorbs light having a predetermined wavelength and receives the incident analysis light L1, adds a light absorption signal to the analysis light L1. The first light receiver 36 receives the analysis light L1 containing the sample derived signal generated by the interaction between the analysis light L1 and the sample 9, the first modulation signal, and the light absorption signal, and outputs the first light reception signal F(t).

The length measuring optical system 4 includes the second light source 41 and the length measuring optical system 40. The second light source 41 outputs the length measurement light L2, which is laser light. The length measuring optical system 40 irradiates the second reflection surface 332 with the length measurement light L2, and acquires a displacement signal corresponding to the position of the moving mirror 33 from the length measurement light L2 reflected off the second reflection surface 332.

The calculation apparatus 7 includes the moving mirror position calculator 72, the light intensity calculator 74, the Fourier transformer 76, and the moving mirror position correction section 78. The moving mirror position calculator 72 generates the moving mirror position signal X(t) based on the displacement signal acquired by the length measuring optical system 4. The light intensity calculator 74 generates the interferogram F(x) (waveform representing intensity of first light reception signal F(t) at each position of moving mirror 33) based on the first light reception signal F(t) and the moving mirror position signal X(t). The Fourier transformer 76 performs Fourier transform on the interferogram F(x) to generate a spectral pattern containing a peak corresponding to the light absorption signal. The moving mirror position correction section 78 calculates a correction value used to correct the moving mirror position signal X(t) based on the position of the peak corresponding to the light absorption signal.

According to the configuration described above, when the position of the moving mirror 33 is measured by using the displacement signal, a correction value used to correct the measured value of the position can be calculated based on the light absorption signal derived from the gas cell 6 in view of the fact that the energy between the levels of the atoms or molecules encapsulated in the gas cell 6 has extremely high precision and stability. That is, the correction value used to precisely measure the position of the moving mirror 33 can be calculated. Therefore, even when the two light reflecting surfaces (first reflection surface 331 and second reflection surface 332) of the moving mirror 33 have poor parallelism, a decrease in the length measurement precision can be compensated. The spectroscopic apparatus 100 can thus generate a high-precision spectral pattern.

In addition, to provide the advantages described above, it is not necessary to provide additional equipment such as a light source thermostatic system even when a small, inexpensive element such as a semiconductor laser element is employed as the second light source 41. The size, weight, power consumption, and cost of the spectroscopic apparatus 100 can thus be reduced.

In the spectroscopic apparatus 100 according to each of the embodiments described above, the first reflection surface 331 is the front surface 335a of the first mirror member 335, and the second reflection surface 332 is the front surface 336a of the second mirror member 336 attached to the rear surface 335b of the first mirror member 335.

According to the configuration described above, in which the moving mirror 33 is configured with the combination of the two mirror members, the reflectance is readily increased at both the first reflection surface 331 and the second reflection surface 332. The configuration described above increases the S/N ratio (signal-to-noise ratio) of the interference light containing the analysis light L1 reflected off the first reflection surface 331. Similarly, the S/N ratio of the interference light containing the length measurement light L2 reflected off the second reflection surface 332 is also increased.

In the spectroscopic apparatus 100 according to each of the embodiments described above, the analysis optical system 3 includes the beam splitter 32 (light divider), which splits the analysis light L1 output from the first light source 51. The gas cell 6 is disposed between the first light source 51 and the beam splitter 32.

According to the configuration described above, when the sample 9 is irradiated with the analysis light L1, the gas cell 6 is also always irradiated with the analysis light L1. The spectroscopic apparatus 100 can therefore be calibrated simultaneously with the acquisition of the spectral pattern, allowing particularly precise spectroscopic analysis.

In the spectroscopic apparatus 100 according to each of the embodiments described above, the analysis optical system 3 includes the beam splitter 32 (light divider), which splits the analysis light L1 output from the first light source 51. The gas cell 6 is disposed between the beam splitter 32 and the first light receiver 36.

According to the configuration described above, when the sample 9 is irradiated with the analysis light L1, the gas cell 6 is also always irradiated with the analysis light L1. The spectroscopic apparatus 100 can therefore be calibrated simultaneously with the acquisition of the spectral pattern, allowing particularly precise spectroscopic analysis.

In the spectroscopic apparatus 100 according to each of the embodiments described above, the length measuring optical system 40 includes the light modulator 444, which shifts the frequency of the length measurement light L2 (laser light) output from the second light source 41. The length measuring optical system 40 acquires a displacement signal corresponding to the position of the moving mirror 33 based on the interference between the length measurement light L2 reflected off the second reflection surface 332 and the length measurement light L2 having a frequency shifted by the light modulator 444.

According to the configuration described above, the displacement of the moving mirror 33 can be captured at intervals sufficiently narrower than the wavelength of the length measurement light L2. A spectral pattern having a wider wavenumber range (wider wavelength range), that is, a spectral pattern having a wider band can thus be acquired.

The spectroscopic apparatus calibration method according to the embodiment described above is a method for calibrating the spectroscopic apparatus 100, which performs spectroscopic analysis of the sample 9, and includes the mirror position measurement step S102, the analysis light radiation step S104, the waveform generation step S106, the Fourier transform step S108, and the correction value calculation step S110. In the mirror position measurement step S102, the gas cell 6 is placed in the optical path of the analysis light L1 in the spectroscopic apparatus 100 according to each of the embodiments described above, and the spectroscopic apparatus 100 is then caused to acquire the displacement signal corresponding to the position of the moving mirror 33 to measure the position of the moving mirror 33. In the analysis light radiation step S104, the analysis light L1 is caused to enter the gas cell 6 with the position of the moving mirror 33 changed, and the first light receiver 36 is caused to receive the analysis light L1 output from the gas cell 6, and output the first light reception signal F(t). In the waveform generation step S106, the interferogram F(x) (waveform indicating intensity of first light reception signal F(t) derived from gas cell 6 at each position of moving mirror 33) is generated based on the first light reception signal F(t) derived from the gas cell 6 and the measured value of the position of the moving mirror 33. In the Fourier transform step S108, Fourier transform is performed on the interferogram F(x) to generate a spectral pattern containing a peak corresponding to the light absorption signal. In the correction value calculation step S110, a correction value used to correct the measured value of the position of the moving mirror 33 is calculated based on a difference between the wavelength at which the peak corresponding to the light absorption signal is located and the fundamental wavelength absorbed by the gas cell 6.

According to the configuration described above, when the position of the moving mirror 33 is measured by using the displacement signal, a correction value used to correct the measured value of the position can be calculated based on the light absorption signal derived from the gas cell 6 in view of the fact that the energy between the levels of the atoms or molecules encapsulated in the gas cell 6 has extremely high precision and stability. That is, the correction value used to precisely measure the position of the moving mirror 33 can be calculated. Therefore, even when the two light reflecting surfaces (first reflection surface 331 and second reflection surface 332) of the moving mirror 33 have poor parallelism, a decrease in the length measurement precision can be compensated. The spectroscopic apparatus 100 can therefore be calibrated so as to generate a high-precision spectrum pattern.

The spectroscopic method according to the embodiment described above is a method for performing spectroscopic analysis of the sample 9, and includes the calibration step S100 and the spectral information correction step S112. In the calibration step S100, the spectroscopic apparatus calibration method according to the embodiment described above is executed. In the spectral information correction step S112, the sample 9 is placed in the optical path of the analysis light L1 in the spectroscopic apparatus 100, and then the spectral pattern containing the information derived from the sample 9 is acquired and corrected based on the correction value.

According to the configuration described above, even when the two light reflecting surfaces (first reflection surface 331 and second reflection surface 332) of the moving mirror 33 has poor parallelism, a decrease in the length measuring precision can be compensated, so that a high-precision spectral pattern can be generated.

As described above, the spectroscopic apparatus, the spectroscopic apparatus calibration method, and the spectroscopic method according to the present disclosure have been described based on the preferable embodiments shown in the drawings, but the embodiments of the present disclosure are not limited thereto. For example, the configuration of each section in the embodiments described above may be replaced with any configuration having the same function, or any other constituent element may be added to the embodiments described above. Furthermore, two or more of the embodiments described above may be combined with each other.

The spectroscopic apparatus calibration method and the spectroscopic method according to the present disclosure may include a step for any purpose added to the embodiments described above.

A Michelson interference optical system is used in the embodiments described above, and other types of interference optical systems may be used.

Furthermore, the arrangement of the sample is not limited to that shown in the drawings. Since the sample derived signal is generated by causing the analysis light to react with the sample, the sample is not necessarily placed at the position described above and may be disposed at any position where the analysis light emitted from the sample enters the first light receiver.

What is claimed is:

1. A spectroscopic apparatus comprising an analysis optical system, a length measuring optical system, and a calculation apparatus and performing spectroscopic analysis of a sample, wherein
the analysis optical system includes
a moving mirror having a first reflection surface configured to reflect analysis light output from a first light source and add a first modulation signal to the analysis light, and a second reflection surface located at a side opposite the first reflection surface, the moving mirror configured to be translated,
a gas cell configured to encapsulate a gas that absorbs light having a predetermined wavelength, and add a light absorption signal to the analysis light, and
a first light receiver configured to receive the analysis light containing sample derived signal generated by a reaction between the analysis light and the sample, the first modulation signal, and the light absorption signal, and output a first light reception signal,
the length measuring optical system configured to irradiate the second reflection surface with the laser light and acquire a displacement signal corresponding to a position of the moving mirror from the laser light reflected off the second reflection surface includes
a second light source configured to output laser light, and
the calculation apparatus configured to
generate a moving mirror position signal based on the displacement signal,
generate a waveform indicating an intensity of the first light reception signal at each position of the moving mirror based on the first light reception signal and the moving mirror position signal,
perform Fourier transform on the waveform to generate a spectral pattern containing a peak corresponding to the light absorption signal, and
calculate a correction value used to correct the moving mirror position signal based on a position of the peak.

2. The spectroscopic apparatus according to claim 1, wherein
the first reflection surface is a front surface of a first mirror member, and
the second reflection surface is a front surface of a second mirror member attached to a rear surface of the first mirror member.

3. The spectroscopic apparatus according to claim 1, wherein
the analysis optical system includes a light divider configured to divide the analysis light output from the first light source, and
the gas cell is placed between the first light source and the light divider.

4. The spectroscopic apparatus according to claim 1, wherein
the analysis optical system includes a light divider configured to divide the analysis light output from the first light source, and
the gas cell is placed between the light divider and the first light receiver.

5. The spectroscopic apparatus according to claim 1, wherein
the length measuring optical system includes a light modulator configured to shift a frequency of the laser light output from the second light source, and
the length measuring optical system is configured to acquire the displacement signal based on interference between the laser light reflected off the second reflection surface and the laser light having the frequency shifted by the light modulator.

6. A spectroscopic apparatus calibration method for calibrating a spectroscopic apparatus configured to perform spectroscopic analysis of a sample, the method comprising:
in the spectroscopic apparatus according to claim 1, placing the gas cell in an optical path of the analysis light, causing the spectroscopic apparatus to acquire the displacement signal, and measuring the position of the moving mirror;
causing the analysis light to enter the gas cell while changing the position of the moving mirror, causing the first light receiver to receive the analysis light output from the gas cell, and outputting the first light reception signal derived from the gas cell;
generating a waveform indicating the intensity of the first light reception signal derived from the gas cell at each position of the moving mirror based on the first light reception signal derived from the gas cell and a measured value of the position of the moving mirror;
performing Fourier transform on the waveform derived from the gas cell to generate a spectral pattern containing a peak corresponding to the light absorption signal; and
calculating a correction value used to correct the measured value of the position of the moving mirror based on a difference between a wavelength at which the peak is located and a basic wavelength absorbed by the gas cell.

7. A spectroscopic method for performing spectroscopic analysis of a sample, the method comprising:
executing the spectroscopic apparatus calibration method according to claim 6; and
in the spectroscopic apparatus, placing the sample in the optical path of the analysis light, then acquiring a spectral pattern containing information derived from the sample, and correcting the spectral pattern containing the information derived from the sample based on the correction value.
